SEQUENCE LISTING (38 sequences)

SEQ ID NO: 1 - RNA, 24 nt, Homo sapiens
  source 1..24: mol_type = other RNA
tccctgagaccctttaacctgtga

SEQ ID NO: 2 - RNA, 22 nt, Homo sapiens
  source 1..22: mol_type = other RNA
acagcaggcacagacaggcagt

SEQ ID NO: 3 - RNA, 22 nt, Homo sapiens
  source 1..22: mol_type = other RNA
cagtgcaatgttaaaagggcat

SEQ ID NO: 4 - RNA, 22 nt, Homo sapiens
  source 1..22: mol_type = other RNA
ttccctttgtcatcctatgcct

SEQ ID NO: 5 - RNA, 22 nt, Homo sapiens
  source 1..22: mol_type = other RNA
tcgtaccgtgagtaataatgcg

SEQ ID NO: 6 - RNA, 23 nt, Homo sapiens
  source 1..23: mol_type = other RNA
tgtaaacatcctacactctcagc

SEQ ID NO: 7 - RNA, 24 nt, Homo sapiens
  source 1..24: mol_type = other RNA
ttaatgctaatcgtgataggggtt

SEQ ID NO: 8 - RNA, 22 nt, Homo sapiens
  source 1..22: mol_type = other RNA
tctcccaacccttgtaccagtg

SEQ ID NO: 9 - RNA, 22 nt, Homo sapiens
  source 1..22: mol_type = other RNA
ctggtacaggcctgggggacag

SEQ ID NO: 10 - RNA, 23 nt, Homo sapiens
  source 1..23: mol_type = other RNA
taccctgtagaaccgaatttgtg

SEQ ID NO: 11 - RNA, 21 nt, Homo sapiens
  source 1..21: mol_type = other RNA
cattattacttttggtacgcg

SEQ ID NO: 12 - RNA, 22 nt, Homo sapiens
  source 1..22: mol_type = other RNA
cattgcacttgtctcggtctga

SEQ ID NO: 13 - RNA, 23 nt, Homo sapiens
  source 1..23: mol_type = other RNA
caaagtgctgttcgtgcaggtag

SEQ ID NO: 14 - RNA, 23 nt, Homo sapiens
  source 1..23: mol_type = other RNA
tgtgcaaatccatgcaaaactga

SEQ ID NO: 15 - RNA, 23 nt, Homo sapiens
  source 1..23: mol_type = other RNA
taaagtgcttatagtgcaggtag

SEQ ID NO: 16 - RNA, 23 nt, Homo sapiens
  source 1..23: mol_type = other RNA
tgtgcaaatctatgcaaaactga

SEQ ID NO: 17 - RNA, 23 nt, Homo sapiens
  source 1..23: mol_type = other RNA
caaagtgcttacagtgcaggtag

SEQ ID NO: 18 - RNA, 23 nt, Homo sapiens
  source 1..23: mol_type = other RNA
aacattcaacgctgtcggtgagt

SEQ ID NO: 19 - RNA, 22 nt, Homo sapiens
  source 1..22: mol_type = other RNA
tagcagcacgtaaatattggcg

SEQ ID NO: 20 - RNA, 72 nt, Homo sapiens
  source 1..72: mol_type = other RNA
cttgggaatggcaaggaaaccgttaccattactgagtttagtaatggtaatggttctcttgctatacccaga

SEQ ID NO: 21 - RNA, 20 nt, Homo sapiens
  source 1..20: mol_type = other RNA
tacagtatagatgatgtact

SEQ ID NO: 22 - RNA, 23 nt, Homo sapiens
  source 1..23: mol_type = other RNA
tttggcactagcacatttttgct

SEQ ID NO: 23 - RNA, 22 nt, Homo sapiens
  source 1..22: mol_type = other RNA
tatggcactggtagaattcact

SEQ ID NO: 24 - RNA, 24 nt, Homo sapiens
  source 1..24: mol_type = other RNA
tggaagactagtgattttgttgtt

SEQ ID NO: 25 - RNA, 22 nt, Homo sapiens
  source 1..22: mol_type = other RNA
tggttgaccatagaacatgcgc

SEQ ID NO: 26 - RNA, 22 nt, Homo sapiens
  source 1..22: mol_type = other RNA
tggagtgtgacaatggtgtttg

SEQ ID NO: 27 - RNA, 22 nt, Homo sapiens
  source 1..22: mol_type = other RNA
tgtcagtttgtcaaatacccca

SEQ ID NO: 28 - RNA, 22 nt, Homo sapiens
  source 1..22: mol_type = other RNA
agagctgagactagaaagccca

SEQ ID NO: 29 - RNA, 23 nt, Homo sapiens
  source 1..23: mol_type = other RNA
tctacagtgcacgtgtctccagt

SEQ ID NO: 30 - RNA, 23 nt, Homo sapiens
  source 1..23: mol_type = other RNA
tgtagtgtttcctactttatgga

SEQ ID NO: 31 - RNA, 22 nt, Homo sapiens
  source 1..22: mol_type = other RNA
tccgtctcagttactttatagc

SEQ ID NO: 32 - RNA, 21 nt, Homo sapiens
  source 1..21: mol_type = other RNA
ctgacctatgaattgacagcc

SEQ ID NO: 33 - RNA, 23 nt, Homo sapiens
  source 1..23: mol_type = other RNA
taaggtgcatctagtgcagatag

SEQ ID NO: 34 - RNA, 24 nt, Homo sapiens
  source 1..24: mol_type = other RNA
aacaacaaaatcactagtcttcca

SEQ ID NO: 35 - RNA, 21 nt, Homo sapiens
  source 1..21: mol_type = other RNA
tagcagcacagaaatattggc

SEQ ID NO: 36 - RNA, 21 nt, Homo sapiens
  source 1..21: mol_type = other RNA
ttgtgcttgatctaaccatgt

SEQ ID NO: 37 - RNA, 21 nt, Homo sapiens
  source 1..21: mol_type = other RNA
taaagtgctgacagtgcagat

SEQ ID NO: 38 - RNA, 22 nt, Homo sapiens
  source 1..22: mol_type = other RNA
tcctgtactgagctgccccgag

(19) United States
(12) Patent Application Publication
(10) Pub. No.: US 2026/0308526 A1
(43) Pub. Date: Oct. 8, 2026

(54) EXTRACELLULAR VESICLES

(71) Applicant: VivaZome Therapeutics Pty Ltd, Belmont, Victoria (AU)

(72) Inventors: David HAYLOCK, Lockleys (AU); Ian NISBET, Ivanhoe, Victoria (AU); Xenia SANGO, Lower Plenty, Victoria (AU); Louise WHITE, Fitzroy North, Victoria (AU); Craig NEWTON, Belmont, Victoria (AU)

(21) Appl. No.: 19/484,919

(22) PCT Filed: May 17, 2024
(86) PCT No.: PCT/AU2024/050509

§ 371 Date: Nov. 17, 2025

(30) Foreign Application Priority Data
May 18, 2023 (AU) 2023901542

(51) Int. Cl. C12N 5/077; A61K 35/33; C12N 15/113

(52) U.S. Cl. CPC C12N 5/0656; A61K 35/33; C12N 15/113; C12N 2310/141

(57) ABSTRACT

The present disclosure relates to microRNAs (miRs) and extracellular vesicles comprising same and therapeutic uses thereof, for example, in modulating angiogenesis.

This application claims priority from Australian Application No. 2023901542 filed on 18 May 2023, the contents of which are to be taken as incorporated herein by this reference.

### TECHNICAL FIELD

The present disclosure relates to microRNAs (miRs), especially to miRs found in extracellular vesicles from gingival fibroblasts and therapeutic uses of the same.

### BACKGROUND OF INVENTION

Angiogenesis is the process through which new blood vessels are formed from pre-existing ones.

Angiogenesis, defined as the formation of new blood vessels from a pre-existing vascular network, naturally occurs in an organism during growth and development and also in response to injury to restore a tissue's blood supply and promote wound healing. The new vessels can be formed by either sprouting angiogenesis, where endothelial cells form sprouts that grow toward an angiogenic stimulus, or intussusceptive angiogenesis, where interstitial tissues invade the existing vessels and form transvascular tissue pillars that expand and split the vessel.

Sprouting angiogenesis comprises several steps: enzymatic degradation of the vessel's basement membrane, endothelial cell proliferation, migration, sprouting, branching, and tube formation. The stabilization and maturation of the newly formed vascular structures require the recruitment of pericytes, the deposition of extracellular matrix, and mechanical stimulation by the shear stress.

In healthy tissues, angiogenesis is tightly regulated by a precise balance between stimulatory and inhibitory signals. Abnormal blood vessel growth occurs when this balance is disturbed and is a major cause of numerous diseases, such as cancer, atherosclerosis, corneal neovascularization, rheumatoid arthritis, or ischemic diseases.

### SUMMARY OF INVENTION

The present inventors have surprising demonstrated that extracellular vesicles derived from human gingival fibroblasts comprise pro- and anti-angiogenic microRNAs (miRNAs or miRs) and that the extracellular vesicles can be used to manipulate angiogenesis.

Accordingly, the present disclosure provides a population of gingival fibroblast derived extracellular vesicles, for example, exosomes.

In one embodiment of the disclosure, the extracellular vesicles are allogeneic.

In one or a further embodiment of the disclosure, the extracellular vesicles are derived from primary gingival fibroblasts. In another embodiment, the extracellular vesicles are derived from gingival fibroblasts expanded in culture. In another embodiment, the extracellular vesicles are derived from an immortalized gingival fibroblast cell line. In some embodiments of the disclosure, the population of extracellular vesicles are derived from gingival fibroblasts cultured under normoxic conditions. In alternative embodiments of the disclosure, the population of extracellular vesicles are derived from gingival fibroblasts cultured under hypoxic conditions.

In some embodiments, the extracellular vesicles may comprise one or more microRNAs selected from miR-125a-5p, miR-214-3p, miR-130a-3p, miR-204-5p, miR-126-3p, miR-30c-5p, miR-155-5p, miR-150-5p, miR-150-3p, miR-10b-5p, miR-126-5p, miR-25-3p, miR-93-5p, miR-19b-3p, miR-20a-5p, miR-19a-3p, and miR-17-5p. For example, the extracellular vesicles may comprise one or more microRNAs, wherein the one or more microRNAs comprise a nucleotide sequence shown in any one of SEQ ID Nos: 1 to 17. Such vesicles may be useful in pro-angiogenic therapeutic applications.

In some embodiments of the disclosure, the extracellular vesicles comprise at least miR-125a-5p, miR-214-3p, miR-130a-3p, miR-204-5p, miR-126-3p, or miR-30c-5p. For example, the extracellular vesicles comprise at least miR-126-3p.

In some embodiments of the disclosure, the extracellular vesicles comprise at least miR-30c-5p, miR-155-5p, or miR-150-5p. For example, the extracellular vesicles may comprise miR-30c-5p in combination with one or more of miR-155-5p, miR150-5p, miR-150-3p, miR-10b-5p, miR-126-5p, miR-126-3p, miR-25-3p, miR-93-5p, miR-19b-3p, miR-20a, miR-19a-3p, and miR-17-5p. In another example, the extracellular vesicles may comprise miR-155-5p in combination with one or more of miR-30c-5p, miR-150-5p, miR-150-3p, miR-10b-5p, miR-126-5p, miR-126-3p, miR-25-3p, 93-5p, miR-19b-3p, miR-20a, miR-19a-3p, and miR-17-5p. In another example, the extracellular vesicles may comprise miR-150-5p in combination with one or more of miR-30c-5p, miR155-5p, miR-150-3p, miR-10b-5p, miR-126-5p, miR-126-3p, miR-25-3p, miR-93-5p, miR-19b-3p, miR-20a, miR-19a-3p, and miR-17-5p.

In some embodiments of the disclosure, the extracellular vesicles comprise at least miR-30c-5p, miR-155-5p, and miR150-5p. For example, the extracellular vesicles may comprise miR-30c-5p, miR-155-5p, and miR150-5p in combination with 126-5p. In another example, the extracellular vesicles may comprise miR-30c-5p, miR-155-5p, and miR150-5p in combination with one or more of miR-125a-5p, miR-214-3p, miR-130a-3p, miR-204-5p, or miR-126-3p.

In some or further embodiments of the disclosure, the extracellular vesicles comprise one or more microRNAs selected from miR181a-5p, miR16-5p, miR-451a, miR-144-3p, miR-96-5p, miR-183-5p, and miR-7-5p. For example, the extracellular vesicles may comprise one or more microRNAs, wherein the one or more microRNAs comprise a nucleotide sequence shown in any one of SEQ ID Nos: 18 to 24. Such vesicles may be useful in anti-angiogenic therapeutic applications.

In some or further embodiments of the disclosure, the extracellular vesicles comprise one or more microRNAs selected from miR-380-5p, miR-122-5p, miR-223-3p, miR-3160-3p, miR-139-5p, miR-142-3p, miR-340-3p, miR-192-5p, miR-18a-5p, miR-3529-3p, miR-195-5p, miR-218-5p, miR-106b-5p, and miR-486-5p. For example, the extracellular vesicles may comprise one or more microRNAs, wherein the one or more microRNAs comprise a nucleotide sequence shown in any one of SEQ ID Nos: 25 to 38.

The present disclosure also provides a population of extracellular vesicles, wherein the extracellular vesicles comprise one or more microRNAs selected from miR-30c-5p, miR-10b-5p, miR-126-5p, miR-25-3p, miR-93-5p, miR-19b-3p, miR-20a, miR-19a-3p, and miR-17-5p.

In one embodiment of the disclosure, the extracellular vesicles also comprise one or more microRNAs selected from miR16-5p, miR-451a, and miR-7-5p.

In one or a further embodiment, the extracellular vesicles also comprise one or more microRNAs selected from miR-139-5p, miR-142-3p, and miR-18a-5p.

In some embodiments of the disclosure, the population of extracellular vesicles are derived from gingival fibroblasts cultured under normoxic conditions.

The present disclosure also provides a population of extracellular vesicles, wherein the extracellular vesicles comprise one or more microRNAs selected from miR-130a-3p, miR-126-3p, miR-150-5p, miR-10b-5p, and miR-126-5p.

In one embodiment of the disclosure, the extracellular vesicles may also comprise one or more microRNAs selected from miR-25-3, miR-93-5p, miR-19b-3p, miR-20a-5p, miR-19a-3p, and miR-17-5p.

In one or a further embodiment of the disclosure, the extracellular vesicles may also comprise one or both microRNAs selected from miR-451a and miR-144-3p.

In one or a further embodiment of the disclosure, the extracellular vesicles may also comprise one or more microRNAs selected from miR-139-5p, miR-142-3p, miR-18a-5p, miR-3529-3p, miR-195-5p, and miR-486-5p.

In one or a further embodiment of the disclosure, the extracellular vesicles may also comprise miR-106b-5p.

In some embodiments of the disclosure, the population of extracellular vesicles is derived from gingival fibroblasts cultured under hypoxic conditions.

In some embodiments of the disclosure, the ratio of extracellular vesicle microRNAs to cell lysate microRNAs is at least 2:1, or at least 5:1, preferably at least 10:1 or more.

For example, the ratio of extracellular vesicle microRNAs selected from one or more of miR-130a, miR-17-5p, miR-19a-3p, miR-20a-5p, miR-19b-3p, miR-7-5p, miR-486-5p, miR-106b-5p, miR-93-5p, miR-25-3p, miR-218-5p, miR-195-5p, miR-3529-3p, miR-18a-5p, miR-192-5p, miR-126-3p, miR-126-5p, miR-183-5p, miR-96-5p, miR-340-3p, miR-10b-5p, miR-150-3p, miR-150-5p, miR-142-3p, miR-144-3p, miR-139-5p, and miR-3160-3p to cell lysate microRNAs is at least 2:1, or at least 5:1, preferably at least 10:1.

In some embodiments of the disclosure, the ratio of extracellular vesicle microRNAs selected from one or more of miR-223-3p, miR-155-5p, miR-122-5p, miR-451a, miR-380-5p, miR-16-5p, miR-181a-5p, and miR-30c-5p to cell lysate microRNAs is at least 50:1, or at least 60:1, or at least 70:1, or at least 80:1, or at least 90:1 or more, preferably 100:1. The ratio of extracellular vesicle microRNAs selected from miR-16-5p, miR-181a-5p, and miR-30c-5p to cell lysate microRNAs may be at least 100:1, or at least 200:1, or at least 300:1, or at least 400:1 or more, preferably at least 500:1.

In some embodiments of the disclosure, at least 50% of the extracellular vesicles have a particle size of about 30 nm to about 150 nm.

In some embodiments of the disclosure, the population promotes angiogenesis.

In some embodiments of the disclosure, the population reduces fibrosis.

In some embodiments of the disclosure, the population slows the rate of progression of fibrosis.

The present disclosure also provides a composition comprising gingival fibroblast derived extracellular vesicles, or a population of extracellular vesicles, according to the disclosure, and one or more pharmaceutically acceptable carriers or excipients.

The present disclosure also provides gingival fibroblast extracellular vesicles, or a population of extracellular vesicles, or a composition, according to the disclosure for use in therapy. For example, for use in the promotion of wound healing, in the treatment or prevention of ischemic diseases, or in the treatment or prevention of fibrotic diseases.

The present disclosure also provides a method for promoting wound healing in a subject, the method comprising administering an effective amount of gingival fibroblast derived extracellular vesicles, or a population of extracellular vesicles, or a composition, according to the disclosure to the wound.

The present disclosure also provides a method for promoting tissue repair and/or regeneration in a subject comprising administering an effective amount of gingival fibroblast derived extracellular vesicles, or a population of extracellular vesicles, or a composition, according to the disclosure to the subject.

The present disclosure also provides a method of treating or preventing an ischemic disease in a subject comprising administering an effective amount of gingival fibroblast derived extracellular vesicles, or a population of extracellular vesicles, or a composition according to the disclosure to the subject.

The present disclosure also provides a method of treating or preventing fibrotic disease in a subject comprising administering an effective amount of gingival fibroblast derived extracellular vesicles, or a population of extracellular vesicles, or a composition, according to the disclosure to the subject.

The present disclosure also provides the use of gingival fibroblast derived extracellular vesicles, or a population of extracellular vesicles, or a composition according to the disclosure, in the manufacture of a medicament for use in therapy. For example, for use in the promotion of wound healing, in the treatment or prevention of ischemic diseases, or in the treatment or prevention of fibrotic diseases.

The present disclosure is not to be limited in scope by the specific examples described herein, which are intended for the purpose of exemplification only. Functionally-equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### BRIEF DESCRIPTION OF DRAWINGS

FIG. 1. ZetaView analysis of cell culture supernatant samples collected from GFD003 (A) and HuGF (B) cell lines.

FIG. 2. Transmission electron micrographs of exosomes produced by gingival fibroblasts.

FIG. 3. Heatmap showing expression of micro-RNA species in gingival fibroblast (cell lysates) and their exosomes.

FIG. 4. Phase contrast micrograph of induced pluripotent stem cell (iPSC)-derived endothelial cells (iCells) 5 days after culture in a 1:10 dilution of concentrated gingival fibroblast exosome sample.

FIG. 5. Phase contrast micrograph of iCell endothelial cells 5 days after culture in 20 ng/ml of VEGF.

FIG. 6. Effect of exosomes from HuGF gingival fibroblasts, in iCell proliferation assay at day 5.

FIG. 7. Effect of exosomes from GFD003 gingival fibroblasts, in iCell proliferation assay at day 5.

Sequence Listing

| Human miRNA | Mature miRNA Sequence | Accession Number | SEQ ID NO: |
|---|---|---|---|
| miR-125a-5p | UCCCUGAGACCCUUUAACCUGUGA | MIMAT0000443 | 1 |
| miR-214-3p | ACAGCAGGCACAGACAGGCAGU | MIMAT0000271 | 2 |
| miR-130a-3p | CAGUGCAAUGUUAAAAGGGCAU | MIMAT0000425 | 3 |
| miR-204-5p | UUCCCUUUGUCAUCCUAUGCCU | MIMAT0000265 | 4 |
| miR-126-3p | UCGUACCGUGAGUAAUAAUGCG | MIMAT0000445 | 5 |
| miR-30c-5p | UGUAAACAUCCUACACUCUCAGC | MIMAT0000244 | 6 |
| miR-155-5p | UUAAUGCUAAUCGUGAUAGGGGUU | MIMAT0000646 | 7 |
| miR-150-5p | UCUCCCAACCCUUGUACCAGUG | MIMAT0000451 | 8 |
| miR-150-3p | CUGGUACAGGCCUGGGGGACAG | MIMAT0004610 | 9 |
| miR-10b-5p | UACCCUGUAGAACCGAAUUUGUG | MIMAT0000254 | 10 |
| miR-126-5p | CAUUAUUACUUUUGGUACGCG | MIMAT0000444 | 11 |
| miR-25-3p | CAUUGCACUUGUCUCGGUCUGA | MIMAT0000081 | 12 |
| miR-93-5p | CAAAGUGCUGUUCGUGCAGGUAG | MIMAT0000093 | 13 |
| miR-19b-3p | UGUGCAAAUCCAUGCAAAACUGA | MIMAT0000074 | 14 |
| miR-20a-5p | UAAAGUGCUUAUAGUGCAGGUAG | MIMAT0000075 | 15 |
| miR-19a-3p | UGUGCAAAUCUAUGCAAAACUGA | MIMAT0000073 | 16 |
| miR-17-5p | CAAAGUGCUUACAGUGCAGGUAG | MIMAT0000070 | 17 |
| hsa-miR-181a- | AACAUUCAACGCUGUCGGUGAGU | MIMAT0000256 | 18 |
| 5p | | | |
| miR-16-5p | UAGCAGCACGUAAAUAUUGGCG | MIMAT0000069 | 19 |
| miR-451a | CUUGGGAAUGGCAAGGAAACCGUUA | MI0001729 | 20 |
| | CCAUUACUGAGUUUAGUAAUGGUA | | |
| | AUGGUUCUCUUGCUAUACCCAGA | | |
| miR-144-3p | UACAGUAUAGAUGAUGUACU | MIMAT0000436 | 21 |
| miR-96-5p | UUUGGCACUAGCACAUUUUUGCU | MIMAT0000095 | 22 |
| miR-183-5p | UAUGGCACUGGUAGAAUUCACU | MIMAT0000261 | 23 |
| miR-7-5p | UGGAAGACUAGUGAUUUUGUUGUU | MIMAT0000252 | 24 |
| miR-380-5p | UGGUUGACCAUAGAACAUGCGC | MIMAT0000734 | 25 |
| miR-122-5p | UGGAGUGUGACAAUGGUGUUUG | MIMAT0000421 | 26 |
| miR-223-3p | UGUCAGUUUGUCAAAUACCCCA | MIMAT0000280 | 27 |
| miR-3160-3p | AGAGCUGAGACUAGAAAGCCCA | MIMAT0015034 | 28 |
| miR-139-5p | UCUACAGUGCACGUGUCUCCAGU | MIMAT0000250 | 29 |
| miR-142-3p | UGUAGUGUUUCCUACUUUAUGGA | MIMAT0000434 | 30 |
| miR-340-3p | UCCGUCUCAGUUACUUUAUAGC | MIMAT0000750 | 31 |
| miR-192-5p | CUGACCUAUGAAUUGACAGCC | MIMAT0000222 | 32 |
| miR-18a-5p | UAAGGUGCAUCUAGUGCAGAUAG | MIMAT0000072 | 33 |
| miR-3529-3p | AACAACAAAAUCACUAGUCUUCCA | MIMAT0022741 | 34 |
| miR-195-5p | UAGCAGCACAGAAAUAUUGGC | MIMAT0000461 | 35 |
| miR-218-5p | UUGUGCUUGAUCUAACCAUGU | MIMAT0000275 | 36 |
| miR-106b-5p | UAAAGUGCUGACAGUGCAGAU | MIMAT0000680 | 37 |
| miR-486-5p | UCCUGUACUGAGCUGCCCCGAG | MIMAT0002177 | 38 |

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise.

Throughout the description and claims of the specification, the word “comprise” and variations of the word, such as “comprising” and “comprises”, is not intended to exclude other additives, components, integers or steps.

Unless specifically defined otherwise, all technical and scientific terms used herein shall be taken to have the same meaning as commonly understood by one of ordinary skill in the art.

All publications cited herein are entirely incorporated herein by reference. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987 including all updates until present); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994 including all updates until present); Colligan et al., Current Protocols in Protein Science, John Wiley & Sons, NY, N.Y., (1997 including all updates until present).

A reference herein to a publication which is given as prior art is not to be taken as admission that publication was known or that the information it contains was part of the common general knowledge as at the priority date of any of the claims.

As used herein, “gingival fibroblasts” refers to cells from gingival tissue. Human gingival fibroblasts (HuGnF) express a wide variety of surface molecules including, for example, CD9, CD26, CD44, CD55, CD59, CD63, CD71, CD73, CD86, CD90, CD95, CD99, CD105, CD117, CD166. They also express mRNAs for protease-activated receptor-1 (PAR-1) and PAR-3. The term as used herein refers to primary cells or cultured primary cells or cell lines derived from gingival fibroblasts (for example, gingival fibroblasts immortalized by ectopic expression of the catalytic domain of human telomerase reverse transcriptase (hTERT) alone or in combination with overexpression of cyclin-dependent kinase-4 or 6 (Cdk4/6); or gingival fibroblasts immortalized by overexpression of c-Myc). In one embodiment, gingival fibroblasts of the disclosure express one or more of CD44, CD73, CD90, CD105 and CD166. In a further embodiment, the gingival fibroblasts do not express detectable levels of CD14, CD34, CD45. As used herein “gingival fibroblasts derived extracellular vesicles” refers to vesicles secreted from gingival fibroblasts and includes exosomes and microvesicles. The term is not intended to cover apoptotic bodies. Typically, extracellular vesicles of the invention have a diameter between 30-300 m in size. The diameter of extracellular vesicles can be determined by one of a number of methods accepted by those skilled in the art. For example, by dynamic light scattering, disc centrifugation, nanoparticle tracking analysis (NTA), tunable resistive pulse sensing (TRPS), atomic force microscopy or electron microscopy. In one embodiment, the diameter of the extracellular vesicles is determined by one or both of NTA and electron microscopy. The extracellular vesicles may express one or more surface markers such as tetraspanins CD9, CD63, CD81, the ALG-2-interacting protein X (ALIX), and the tumor susceptibility gene 101 protein (TSG101). The extracellular vesicles of the invention also have a molecular cargo (one or more miRNAs) as defined herein and demonstrate biological function including, but not limited to angiogenic or antifibrotic activity as demonstrated by in vitro and/or in vivo assays and models disclosed herein.

The term “apoptotic bodies” refers to vesicles produced by dying cells having a diameter between 50-5000 nm. Apoptotic bodies contain exposed phosphatidylserine on their membranes, and their major protein markers include histones, TSP, and C3b. A notable distinction between apoptotic bodies and exosomes and microvesicles is that apoptotic bodies also contain fragmented DNA and cell organelles from their host cell.

The term “exosome” refers to cell-derived vesicles having a diameter of between about 30 and 150 nm, preferably a diameter of about 50-150 nm, for example, a diameter of about 60 nm, 70 nm, 80 nm, 90 nm, 100 nm, 110 nm, 120 nm, or 130 nm. In one embodiment, the exosomes of the present disclosure have an average diameter of about 90 nm as determined by NTA. In another embodiment, the exosomes of the present disclosure have an average diameter of about 100-130 nm as determined by NTA.

Exosomes include specific surface markers not present in other vesicles, including surface markers such as tetraspanins, for example, CD9, CD37, CD44, CD53, CD56, CD63, CD81, CD82, CD151, and CD271; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LA1VIP2 (lysosome-associated membrane protein) and LIMP (lysosomal integral membrane protein). Normally used exosome markers include ALIX, TSG101, tetraspanins (CD81, CD63, CD9), and flotillin.

The term “microvesicles” refers to cell-derived vesicles having a diameter of between about 50 and 1000 nm, preferably a diameter of about 50-500 nm, for example, a diameter of about 100 nm, 150 nm, 200 nm, 250 nm, 300 nm, 350 nm, 400 nm, 450 nm, or 500 nm. Microvesicles include specific surface markers not present in other vesicles, including surface markers such as integrins, selectins, CD40.

As used herein, the term “angiogenesis” relates to a process which involves the vascularisation of a tissue, in particular, the proliferation, migration and infiltration of vascular endothelial cells and the growth and the development of new capillary blood vessels.

As used herein, “disease”, “disorder”, “condition” and the like, as they relate to a subject's health, are used interchangeably and have meanings ascribed to each and all of such terms.

As used herein, a subject “at risk” of developing a disease or relapse thereof or relapsing may or may not have detectable disease or symptoms of disease and may or may not have displayed detectable disease or symptoms of disease prior to the treatment according to the present disclosure. “At risk” denotes that a subject has one or more risk factors, which are measurable parameters that correlate with development of the disease, as known in the art and/or described herein.

As used herein, the terms “treating”, “treat” or “treatment” include administering gingival fibroblast derived extracellular vesicles, for example, to thereby reduce or eliminate at least one symptom of a specified disease or to slow progression of the disease.

As used herein, the term “preventing”, “prevent” or “prevention” includes providing prophylaxis with respect to occurrence or recurrence of a specified disease. An individual may be predisposed to or at risk of developing the disease or relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment or prevention of a disease as described herein. In one example, the term “effective amount” is meant an amount necessary to promote wound healing, tissue repair, or regeneration. In another example, the term “effective amount” is meant an amount necessary to treat or prevent an ischemic disease. The effective amount may vary according to the disease to be treated or factor to be altered and also according to the weight, age, racial background, sex, health and/or physical condition and other factors relevant to the subject being treated. Typically, the effective amount will fall within a relatively broad range (e.g., a “dosage” range) that can be determined through routine trial and experimentation by a medical practitioner. Accordingly, this term is not to be construed to limit the disclosure to a specific quantity. The effective amount can be administered in a single dose or in a dose repeated once or several times over a treatment period.

A “therapeutically effective amount” is at least the minimum concentration required to effect a measurable improvement of a particular disease. A therapeutically effective amount herein may vary according to factors such as the disease state, age, sex, and weight of the patient. A therapeutically effective amount is also one in which any toxic or detrimental effects of treatment are outweighed by the therapeutically beneficial effects. In one example, the therapeutically effective amount is the amount of gingival fibroblast derived extracellular vesicles which can promote wound healing, tissue repair, or regeneration. In another example, the therapeutically effective amount is the amount of gingival fibroblast derived extracellular vesicles which can be used to treat or prevent an ischemic disease.

As used herein, the term “prophylactically effective amount” shall be taken to mean a sufficient quantity of gingival fibroblast derived extracellular vesicles to prevent or inhibit or delay the onset of one or more detectable symptoms of a disease or a complication thereof, for example inhibit or delay ischemic disease.

As used herein, the term “subject” shall be taken to mean any mammalian animal, for example, a human with or at risk of ischemic disease.

### Extracellular Vesicles

Extracellular vesicles useful in the methods of the disclosure include exosomes and microvesicles. Exosomes are nanometer-sized vesicles of endocytic origin that form by inward budding of the limiting membrane of multivesicular endosomes. Their size is typically equivalent to that of the intraluminal vesicle within multivesicular endosomes (40-120 nm). Due to their endocytic origin exosomes are commonly enriched in endosome-associated proteins such as Rab GTPases, SNAREs, annexins, and flotillin. Some of these proteins (e.g. ALIX and TSG101) are normally used as exosome markers. Tetraspanins are a family of membrane proteins known to cluster into microdomains at the plasma membrane. These proteins are abundant in exosomes and considered to be markers as well. Microvesicles bud from the cell surface and their size may vary between 50-1,000 nm. Common protein markers used to define these vesicles are selectins, integrins and the CD40 ligand. Extracellular vesicles of the disclosure are isolated from gingival fibroblasts. The extracellular vesicles may comprise one or more miRs as defined herein.

As used herein, a “miR gene product” or “miRNA” or “miR” means the unprocessed or processed RNA transcript from a miR gene. The miR gene products are not translated into proteins, so the term “miR gene product” does not include proteins. Typically the processed miR or “mature” miRNA comprises about 19-25 nucleotides.

As used herein, “miR gene expression” refers to the production of miR gene products from a miR gene, including processing of the miR precursor into a processed (mature) miR gene product.

### Isolation of Gingival Fibroblasts

Numerous methods are known in the art to isolate gingival fibroblasts. One of the earliest descriptions for isolation of human gingival fibroblasts was published in 1977 by Jacobsen (Scand. J Dent Res. (1977) 85 (7): 567-74). Jacobsen described mincing of the gingival tissue, enzymatic digestion with trypsin then culture in minimal essential media with 10% fetal calf serum (FCS). This method is still the basis for most methods used to derive gingival fibroblasts.

One of the first groups to isolate a population of progenitor cells (as distinct from fibroblasts) from gingival tissue, termed gingiva-derived MSCs was Zhang et al., in 2009 (J. Immunol. (2009) 183 (12): 7787-7798). However, the definitive study describing isolation and propagation of clonal populations of gingival fibroblasts from healthy and inflamed gingival tissue was published by the Bartold and Gronthos group in 2012 (Regen. Med. (2012) 7 (6): 819-832).

In one example, gingival tissue is digested using collagenase type I and dispase. Cells are then filtered through a cell strainer, resuspended into alpha-minimal essential medium (or similar) with 10-20% FCS supplemented with various additives, then plated onto cell culture plastic ware at about 50-60 cells/cm². This provides a bulk culture of gingival fibroblasts which are a heterogeneous population of cells. Alternatively, clones of gingival fibroblasts can be generated by culturing cells at much lower numbers/cm². These clones can then be individually harvested, expanded and characterised.

In one embodiment, extracellular vesicles are isolated from a heterogenous population of gingival fibroblasts, not clones.

In one embodiment, primary gingival fibroblasts are derived from healthy gingiva. Preferably the donors are from 18-30 years of age. The primary gingival fibroblasts may be expanded in culture, for example, in serum free culture medium.

In one embodiment, the gingival fibroblasts express one or more surface makers such as CD44, CD73, CD90, CD105 and CD166. In a further embodiment, the gingival fibroblasts do not express CD45, CD34 and CD14.

In another embodiment, extracellular vesicles are isolated from commercially available gingival fibroblasts. Vendors include AcceGen Biotechnology, (Fairfield, New Jersey, USA), ATCC (Virginia USA), ScienCell (Carlsbad, CA) and PROVITRO AG (Berlin).

### Culture of Cells for Extracellular Vesicle Production

In some embodiments, extracellular vesicles are derived from cells that are passaged multiple times, for example, 3-20 passages. The culture method may involve seeding the cells at an appropriate density in a tissue culture vessel and then incubating the cells in a suitable medium or buffer for a suitable period of time. In some embodiments, the cells may be permitted to attach to the culture vessel before the extracellular vesicles are isolated. In other embodiments, the cells may be kept in suspension, for example, if attached to microcarriers. Suitable microcarriers include those composed of a dextran matrix, with diethylaminoethyl (DEAE) anion exchange groups, such as Cytodex 1 (Sigma Aldrich), Cytodex 2 (Sigma Aldrich), Cytodex 3 (Sigma Aldrich) and Hillex (Sartorius). Microcarriers made of biodegradable substances including gelatin (CultiSpher family, (Sigma Aldrich)), collagen, polylactic-co-glycolic acid (PLGA), poly-I-lactic acid (PLLA), and hydroxyapatite may also be used. The cells may be permitted to replicate in culture before the extracellular vesicles are isolated. Alternatively, the extracellular vesicles may be isolated from cells that have not replicated or replicated minimally (e.g. less than 1 doubling). In some embodiments, the cells are incubated for 1, 2, or 3 days before harvesting.

The cells may be seeded in a tissue culture method at a suitable cell density. The cell density (cells per unit area) may range from about 1 k/cm², 5 k/cm², about 10 k/cm², about 15 k/cm², about 20 k/cm², about 25 k/cm², about 30 k/cm², about 35 k/cm², about 40 k/cm², about 45 k/cm², about 50 k/cm², about 55 k/cm², about 60 k/cm², about 70 k/cm², about 75 k/cm². In some embodiments the cell density (cells per unit area) may range from about 1 k/cm²-100 k/cm², 10 k/cm²-90 k/cm², 20 k/cm²-80 k/cm², 30 k/cm²-70 k/cm², 40 k/cm²-60 k/cm². In some embodiments, the cells are seeded at a density (cells per unit area) of 40 k/cm².

The cells may be seeded in any isotonic solution. In one embodiment a suitable solution may include a suitable buffer. Examples of suitable buffers may include phosphate buffered saline (PBS), HEPES and the like. In other embodiments the cells may be seeded in any suitable cell culture medium, many of which are commercially available. Exemplary media include DMEM, RPMI, MEM, Media 199, HAMS, RoosterBio Nourish and Rooster XF media, and the like. In one embodiment the medium is alpha-MEM. The media may be supplemented with one or more of the following: growth factors, cytokines, hormones, serum, such as FCS, serum substitutes such as knock out replacement serum or B27, antibiotics, vitamins and/or small molecule drugs.

The cells may be placed in a suitable environment, such as a cell incubator heated to about 37° C. In some embodiments the cells may be incubated at room temperature. The incubator may be humidified and have an atmosphere that is about 5% CO₂ and about 1% O₂. In some embodiments the CO₂ concentration may range from about 1-20%, 2-10%, 3-5%. In some embodiments the O₂ concentration may range from about 1-20%, 2-10%, 3-5%.

The cells may be incubated in the medium or buffer for about 1-144 hours, 1-72 hours, 1-48 hours, 2-24 hours, 3-18 hours, 4-16 hours, 5-10 hours. In some embodiments, the cells are incubated for about 24 hours. Thermal stress, anoxia, radiation, and pH of the microenvironment can also be used to increase extracellular vesicle production and/or release using standard techniques known in the art.

In one embodiment, the cells are cultured under normoxic conditions. In another embodiment, the cells are cultured under hypoxic conditions.

After incubation of the cells as described above, the conditioned cell culture medium or buffer may be harvested and the extracellular vesicles isolated. For example, the conditioned cell culture medium or buffer may be pipetted or decanted into another vessel such as a centrifuge tube.

### Isolation of Extracellular Vesicles

Numerous methods are known in the art to isolate extracellular vesicles from different samples including conditioned cell culture medium. Once isolated, extracellular vesicles can be characterized by technology such as nanoparticle tracking analysis (NTA), dynamic light scattering, and nanoscale flow cytometry.

In some embodiments, the extracellular vesicles are isolated by centrifugation. For example, particles with a high buoyant density are first sedimented, such as cells, cell debris, apoptotic bodies, and aggregates of biopolymers. In order to reduce losses caused by co-sedimentation and to decrease contamination of the preparations with the products of cell lysis, this step typically includes several substeps, for example, centrifugation at 300-400×g for about 10 min to sediment a main portion of the cells, at 2000×g to remove cell debris, and at 10,000×g to remove the aggregates of biopolymers, apoptotic bodies, and the other structures with the buoyant density higher than that of extracellular vesicles. Extracellular vesicles contained in the resulting supernatant can be sedimented by ultracentrifugation at, for example, >100,000×g (100,000-200,000×g) for about 2 hours. The obtained extracellular vesicle preparation can be further purified and the isolated microparticles selected according to their size by microfiltration of suspension using filters with pore diameters of, for example, 0.1, 0.22, or 0.45 μm.

In alternative embodiments, low-speed centrifugation (<10,000×g) can be used to remove cells and cell debris or centrifugation at 16,000×g. Different spinning speeds (100,000 to 200,000×g) can also be used for final extracellular vesicle sedimentation.

Ultracentrifugation can be used isolate the extracellular vesicle fraction with a size of 20-250 nm. The isolated extracellular vesicles display one or more of the following markers: CD9, CD63, CD81, TSG101, ALIX, Flotillin-1, AQP2, and FLT1.

In some embodiments, density gradient ultracentrifugation is used in order to increase the efficiency of particle separation according to their buoyant density. This method enables separation of subcellular components, such as mitochondria, peroxisomes, and endosomes and is typically used to isolate microvesicles. Density gradient ultracentrifugation utilizes two methods for formation of the gradient, namely, a continuous density gradient (formed either during centrifugation or upfront) or a stepwise gradient (the density increases in a discrete manner), above a sucrose cushion. A long high-speed centrifugation results in concentration of the exosome-like vesicles in a band with close densities (exosomes, approximately 1.1-1.19 g/ml, but varying depending on the extracellular vesicle content); thus, extracellular vesicles can be separated from proteins and nucleoproteins. The extracellular vesicles isolated by ultracentrifugation typically express different exosomal markers, such as CD9, CD63, CD81, TSG101, ALIX, Flotillin-1, AQP2, HSP70, and FLT1 as well as some amount of non-extracellular vesicle proteins.

Differential ultracentrifugation can be used to isolate the extracellular vesicle fraction with a size of 50-100 nm. In some embodiments, the isolated extracellular vesicle fraction does not comprise any vesicles over 200 nm.

There are numerous protocols known in the art for extracellular vesicle isolation that utilize the separation of micro/nanoparticles according to their size, including ultrafiltration, hydrostatic dialysis, and gel filtration.

Commercial membrane filters have pores of various diameters with a narrow range of pore size distribution, which simplifies isolation of the particles with a specified size. In some embodiments, a method used for extracellular isolation can be supplemented with micro- or ultrafiltration. Ultrafiltration may alternate successive ultracentrifugation stages or it can be an additional step to gel filtration chromatography or size exclusion chromatography.

When isolating extracellular vesicles by microfiltration, the filters with pore diameters of 0.8, 0.45, 0.22, and 0.1 μm are typically used; such filters retain the particles with diameters of over 800, 450, 220, and 100 nm, respectively (+/−20%). Larger particles are removed first (by filters with pore diameters of, for example, 0.8 and 0.45 μm) and the particles with a size smaller than the target extracellular vesicles are separated from the filtrate at the next stage (by filters with pore diameters of, for example, 0.22 and 0.1 μm). Thus, the extracellular vesicle fraction of a specified size is concentrated.

Protocols utilizing ultrafiltration in combination with centrifugation and ultracentrifugation can be used to separate individual fractions of large microvesicles and exosomes in a selective manner. Microfiltration through the filters with a pore diameter of, for example, 0.65 μm and centrifugation at, for example, 10,000×g gives microvesicles, while successive filtration using, for example, 0.65, 0.45, 0.22, and 0.1 μm filters and ultracentrifugation allows for selective isolation of exosomes.

The difference in the composition of isolated fractions can be confirmed by cryoelectron microscopy, particle size analysis by dynamic light scattering or NTA, and/or western blot assays for ALIX, TSG101, CD63, CD81, and EpCAM proteins.

Another method for selective isolation of exosomes is the successive ultrafiltration comprising several stages, namely, filtration using, for example, 0.1 μm filter (e.g., Millipore Express (PES) membrane Stericup Filter Unit with a low affinity for proteins) and five-time tangential flow filtration using, for example, 0.1 μm filter (e.g., 100 nm TrackEtch filter, Millipore, United States). The first stage separates the exosomes and microvesicles from the very large particles; tangential flow filtration cleans the specimen from small-sized contaminants (mainly proteins), and the final step selectively separates exosomes and microvesicles.

In other embodiments, the extracellular vesicles can be isolated by gel filtration (size exclusion chromatography). Gel filtration makes it possible to separate the molecules differing in their hydrodynamic radius and is widely used for separation of biopolymers (proteins, polysaccharides, proteoglycans, etc.). Pretreatment and concentration of extracellular vesicle samples by ultracentrifugation or ultrafiltration are typically required in order to obtain the extracellular vesicle preparations free of proteins and lipoprotein impurities.

In some embodiments, extracellular vesicles are isolated by utilising methods that change extracellular vesicle solubility and/or aggregation. Extracellular vesicles can be precipitated using PEG solutions. This method utilizes a decrease in the solubility of compounds in the solutions of superhydrophilic polymers, PEGs. The procedure comprises mixing of the sample and polymer solution, incubation, and sedimentation of extracellular vesicles by low-speed centrifugation (for example, at 1500×g). The extracellular vesicles can be resuspending in, for example, PBS. The size of the extracellular vesicles isolated with PEG is comparable to the particles isolated by ultracentrifugation, ultrafiltration, and gel chromatography.

In some embodiments, a positively charged molecule, for example, protamine, can be used to aggregate and isolate extracellular vesicles. The protamine can be used in combination with PEG, for example PEG 35,000 Da. For example, the sample is first centrifuged (1500-3000×g). Then biological samples are mixed with precipitating solutions (4:1), such as 1-0.1 mg/ml protamine, 0.2 g/ml PEG 35,000, or a mixture of protamine and PEG. The resulting solution is incubated overnight and centrifuged at, for example, 1500×g (30 min, 22° C.). The pellet is then suspended in buffer and gel-filtered on, for example, a Sephadex G-100 (e.g., GE Healthcare Bio-Sciences AB, Sweden) column to purify the sample from lipoproteins, other low molecular weight impurities, and protamine.

In other embodiments, extracellular vesicles are isolated by neutralizing their surface charge with sodium acetate. Sodium acetate is thought to interfere with the hydration of extracellular vesicle surface, compensates the negative charge, and initiates extracellular vesicle aggregation via hydrophobic interactions. For example, the sample is first centrifuged (500×g, 30 min; 12,000×g, 30 min) to remove cells, debris, and large vesicles; then the supernatant is mixed with 0.1 volume of sodium acetate buffer (1.0 M pH 4.75) and incubated on ice for 30-60 min and additionally for 5 min at 37° C. Extracellular vesicles are sedimented by centrifugation (5000×g, 10 min); the pellet is washed with 0.1 M sodium acetate buffer and centrifuged under the same conditions to suspend the pellet in HBS (HEPES buffered saline). The precipitation procedure is repeated if necessary.

In other embodiments, extracellular vesicles are isolated based on precipitation of proteins with an organic solvent, PROSPR (PRotein Organic Solvent PRecipitation) rather than extracellular vesicle precipitation. This method is based on protein precipitation in acetone under conditions that retain hydrophobic vesicles in supernatant. For example, the sample is supplemented with fourfold volume of cold acetone (−20° C.) and centrifuged (3000×g for 1 min) and the supernatant containing extracellular vesicle fraction is concentrated in a vacuum concentrator.

In other embodiments, extracellular vesicles can be affinity purified. Because extracellular vesicles are rich in proteins and contain many receptors on their surfaces, antibodies can be used to purify them. For example, exosomes can be purified using antibodies specific for some of the most common exosomal protein markers, such as: CD9, CD81, CD63, CD82, Hsp70, Ras-related protein Rab-5b, cytoskeletal protein actin and TSG101.

Specific antibodies to extracellular vesicle markers can be used to select desired extracellular vesicle population (immunoenrichment) or to trap unwanted extracellular vesicle populations (negative selection or immunodepletion). Because extracellular vesicles are very heterogeneous in accordance to their origin, abundance of these markers on different extracellular vesicle also varies. So, a combination of specific antibodies can be used to capture different types of extracellular vesicles.

Antibodies covalently bound to the fixed phase are typically used for this purpose. Magnetic beads, highly porous monolithic silica microtips, surface of plastic plates, cellulose filters, and membrane affinity filters are also used for this purpose.

In some embodiments, annexin 5, a protein binding to phosphatidylserine in the presence of calcium ions can be used to isolate extracellular vesicle having phosphatidylserine on their surface. Phosphatidylserine is exposed on the surface of extracellular vesicles, in particular, microvesicles, apoptotic bodies, and, to a less degree, exosomes.

In some embodiments, extracellular vesicles are isolated based on the ability of heparin to bind extracellular vesicles. For example, extracellular vesicles can be isolated from conditioned cell culture medium using an agarose sorbent with heparin, e.g., Affi-Gel® Heparin Gel (Bio-Rad). Binding of heat shock proteins can also be used to isolate extracellular vesicles according to known methods. For example, the peptide venceremin (Vn) can be used.

In other embodiments, lectins can be used to agglutinate extracellular vesicles. Lectins are the proteins that reversibly, noncovalently, and highly specifically bind carbohydrate motifs of glycoproteins, proteoglycans, and glycolipids.

The skilled person would appreciate that the properties of a sample need to be taken into account when using a particular method for isolation of extracellular vesicles, since the protocol should be fit to specific characteristics of the sample, such as viscosity, presence of specific proteins, extracellular concentration, and the type of further analysis/use of the isolated extracellular vesicles. It is well known that different methods can result in different extracellular vesicle subpopulations. Moreover, the extracellular vesicle isolation efficiency by different methods depends on the nature of the sample.

The isolated extracellular vesicles can be resuspended in any suitable buffer or medium for administration to a subject.

Once isolated, the extracellular vesicle preparation can be characterised by one or more of TEM, NTA, dynamic light scattering, flow cytometry, and immunohistochemical analysis for the markers specific of an isolated extracellular vesicle type to characterise the extracellular vesicle morphology, biochemical composition, and the receptors expressed by the vesicles.

### Administration of Extracellular Vesicles

In some embodiments, the extracellular vesicles of the disclosure can be used to promote angiogenesis to, for example, promote wound healing or tissue or organ regeneration. In one embodiment, the wound healing is associated with diabetes and/or critical limb ischemia.

In other embodiments, the extracellular vesicles of the disclosure can be used to promote angiogenesis to, for example, treat or prevent ischemic conditions and disease, or to treat (e.g., reduce or slow progression of) fibrosis.

In some methods of the disclosure, the extracellular vesicles are administered to an animal to treat or prevent ischemic conditions and disease.

Ischemic diseases include chronic limb threatening ischemia (previously referred to as critical limb ischemia), cardiac ischemia, renal ischemia and CNS ischemia, secondary to stroke or surgery.

In other methods, the extracellular vesicles are administered to a subject to treat fibrosis. Renal, hepatic, cardiac, pulmonary, CNS and skin fibrosis are clinical targets for gingival fibroblast extracellular vesicles of the disclosure.

In other methods, the extracellular vesicles are administered to a subject to treat fibrosis to treat peripheral nerve damage to enhance neuronal survival and growth.

The extracellular vesicles may be administered to the animal by any suitable means. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial (e.g., intrathecal or intraventricular): or intraocular administration.

The extracellular vesicles are preferably delivered as a composition. The composition may be formulated for topical, pulmonary, oral or parenteral administration. Compositions may include sterile aqueous solutions which may also contain buffers, diluents and other suitable excipients. In some embodiments, the composition comprises sucrose, trehalose or PVP40. The composition may be freeze dried.

### Examples

### Cells and Collection of Cell Culture Supernatant Samples

The isolation and propagation of clonal populations of gingival fibroblasts (GF) from healthy and inflamed gingival tissue was described by the Bartold and Gronthos group in 2012 (1). The cell culture samples, and the exosomes characterised by the present inventors were all isolated from cultures of GF derived using the Bartold and Gronthos methodology. In brief, gingival tissue was collected by biopsy from consenting adult donors having periodontal procedures. The tissue was diced and digested in collagenase type I (3 mg/ml; Worthington Biochemical Corporation, NJ, USA) and dispase II (neutral protease; 4 mg/mL; Roche Diagnostic, In, USA) for 2 hours at 37° C. then filtered through a 70 μm cell strainer. The single cell suspension was plated at a concentration of 50-60 cells/cm² in 10 cm tissue culture flasks in alpha-minimal essential medium (a-MEM; Sigma-Aldrich, MO, USA) with 10-20% FCS supplemented with various additives including, 2 mM L-glutamine (Sigma-Aldrich), 100 μM L-ascorbate-2-phosphate (Wako Pure Chemicals Industries Ltd, Japan), 1 mM Sodium pyruvate (Sigma-Aldrich), 50 U/mL penicillin G with 50 μg/mL streptomycin (JRH Biosciences Inc., KS, USA) and 2.5 μg/mL amphotericin B (Fungizone; Life Technologies, NY, USA) in a humidified incubator (37° C., 45% CO₂).

Alternatively, single cells were seeded into 25 cm² tissue culture plates for initial culture. This provides a bulk culture of gingival fibroblasts which are a heterogeneous population of cells. Culture medium was changed twice a week. Once the adherent cells become 90% confluent, they can be passaged following enzymatic detachment using 0.05% trypsin/EDTA (Gibco) and quenching with FCS. Prior to replating, the cells were washed twice by centrifugation at 200×g and resuspended in complete media [Minimal Essential Medium Eagles (Alpha Modification) (x-MEM: Sigma, Cat No. M4526-500 ml) supplemented with 10% (v/v) Fetal Bovine Serum (FBS: CellSera Australia Batch No F21701), 2 mM L-glutamine (JRH Biosciences), 100 μM L-ascorbate-2-phosphate (WAKO Pure Chemical Industries, Japan), 50 U/ml penicillin, 50 μg/ml streptomycin (JRH Biosciences)].

These cells were cryopreserved following enzymatic detachment and washing (as described above) then frozen by rate-controlled freezing in 10% DMSO and complete media. Cells were thawed, washed and replated in T-flasks in complete media and used to provide cell culture supernatant samples for exosome analysis. In some cases, the GF cells were cultured in normoxic or hypoxic (5% O₂) conditions. In each case cell culture harvesting was performed when the GF cells were 80-90% confluent as assessed microscopically. Prior to harvesting of cell culture supernatant, cultures were washed twice in a-MEM without any FCS (so as to remove “contaminating” vesicles) and re-cultured in a-MEM alone for 24-48 hours. Cell culture supernatant was then harvested at 24 or 48 hours after re-culture in a-MEM alone.

Following harvesting, cell culture medium was strained through a 70 μM filter and centrifuged at 2,000×g to deposit cells and cellular debris. The “clean” supernatant was decanted and placed into polypropylene tubes and frozen at −80° C.

To enable analysis of the GF cells (for RNA sequencing), cells were collected following thorough washing, then trypsinization. The cells were centrifuged and cell pellets resuspended in ice cold PBS and frozen immediately. The cell culture supernatant samples and cell pellets were then analyzed, including small RNA sequencing to enable comparison of miRNA abundance between exosomes and their parental cells. Samples from two gingival tissue donors, GFD003 and HuGF were processed in this manner and material used for the studies described below.

Immunophenotyping of the passaged GF cells demonstrated that they express CD44, CD73, CD90, CD105 and CD166 but not the leucocyte markers CD34, CD45 and CD14.

### Exosome Analytics

### Exosome Counting and Sizing

The frozen cell culture supernatant sample were thawed and analysed on the same day. The number of vesicles and their size distribution were analysed by nanoparticle tracking analysis using either the Nanosight NS300 (Malvern Panalytical) or ZetaView (Particle Metrix) platforms. For analysis on the Nanosight, samples were diluted to ensure that the particle per frame is between 10-100. For the Zetaview, samples were diluted to ensure 50-200 particles/frame are imaged. In both cases, analysis was performed according to standard operating protocols.

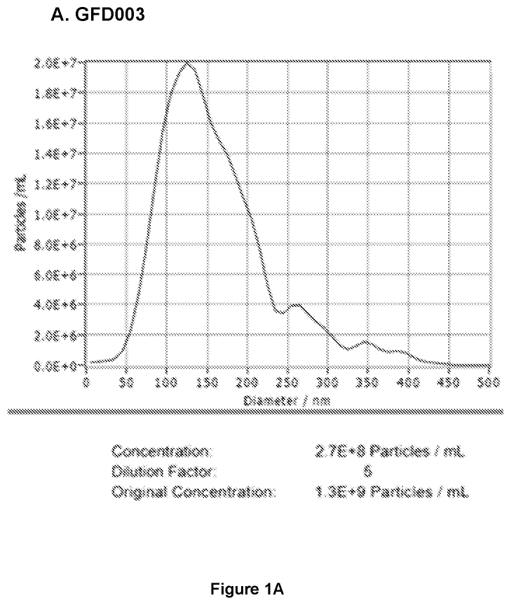

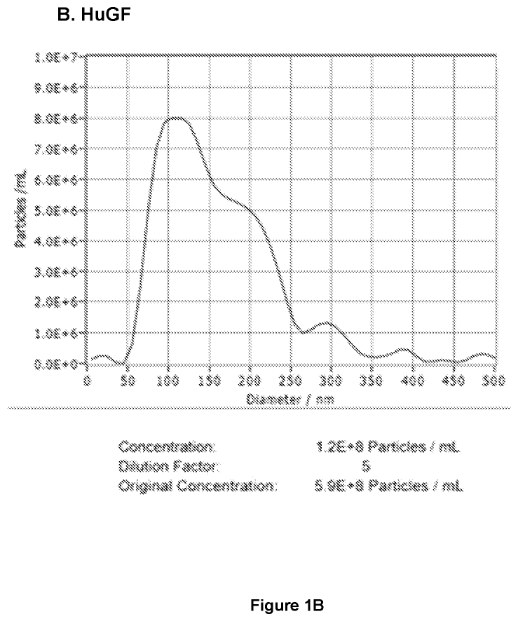

A representative example of analysis of GF cell culture supernatant samples from two GF lines (GFD003 and HuGF) using ZetaView is shown in FIG. 1.

The concentration of exosome particles within gingival fibroblast cell culture samples ranged from 3.00×10⁸ to 1.3×10⁹/mL.

### Transmission Electron Microscopy

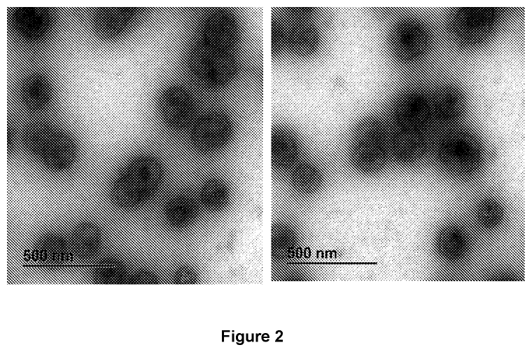

Exosome morphology was assessed by transmission electron microscopy using a Jeol JEM-2100 200 kV analytical electron microscope. In brief, 20 μL of exosome sample was transferred to a clean tube then 20 μL of 2% glutaraldehyde added and allowed to stand at room temperature for 30 minutes. Two×100 μL drops of milliQ water and one 30 μL drop of uranyl acetate (1.5% uranyl acetate/water) per sample was then added. A small volume (5-6 μL) of the fixed and stained sample was then placed onto a glow discharge grid and allowed to absorb for 5 minutes. Excess sample was removed gently and the grid dried before imaging on the Jeol JEM-2100, using a standard protocol. As is evident from the transmission electron microscopy images shown in FIG. 2, the vesicles produced by gingival fibroblasts have a typical exosome cup-shaped morphology with an electron dense central zone.

### Small RNA Sequencing

Small RNA sequencing was performed on gingival fibroblast cells and exosomes isolated from cell culture supernatants to enable a direct comparison of the miRNA species within exosomes as compared to parental cells.

Cell culture supernatant was harvested from gingival fibroblast cultures when they were 80-90% confluent. Two gingival fibroblast samples (Donor 1 and Donor 2) were cultured under normoxic or hypoxic conditions. Cells were harvested after the cell culture supernatant was collected, washed then pelleted to prepare cell lysates. The method used for small RNA sequencing is based on that published by Lesley Cheng and Andrew Hill (3). RNA sequencing first involves construction of a small RNA library to be sequenced on the Ion Torrent S5 device. All reagents used for library construction are provided in the Ion Total RNA-Seg kit version 2 and all procedures were carried out according to strict protocols to ensure RNA did not degrade. In brief, the following steps were followed: RNA isolation and quantification, enrichment of small RNA, hybridisation and ligation of RNA, synthesis of cDNAs for PCR amplification, purification and size selection of cDNA products, amplification of cDNA products by PCR, purification and size selection of PCR products followed by an assessment of the yield and size distribution of the amplified library cDNA.

Cell culture supernatant samples from cultures of gingival fibroblasts were first concentrated by ultracentrifugation with a final centrifugation step at 100,000×g then the exosome rich pellet resuspended in 250 μL of sterile PBS. To this, 750 μL of TRIzol LS was added (rather than standard TRIzol which is only used for cell pellets and samples not resuspended in buffer), vortexed for 5-10 seconds then incubated for 5 minutes at room temperature. Thereafter chloroform extraction was performed to separate the RNA into an aqueous layer for precipitation and concentration of RNA using RNeasy mini columns. The resultant exosomal RNA was quantitated and assessed using the RNA Nano device, by following the manufacturer's instructions. Only samples containing greater than 1% micro-RNA were further processed for construction of cDNA libraries and subject to sequencing.

After library construction, the libraries were templated using a One Touch 2 or Ion Chef system and sequenced on the Ion Torrent Personal Genome Machine (PGM) or Ion S5 benchtop sequencer with the potential to obtain 5-6 million reads per 2318 chip or 80 million reads per 540 chip using 200 basepair sequencing.

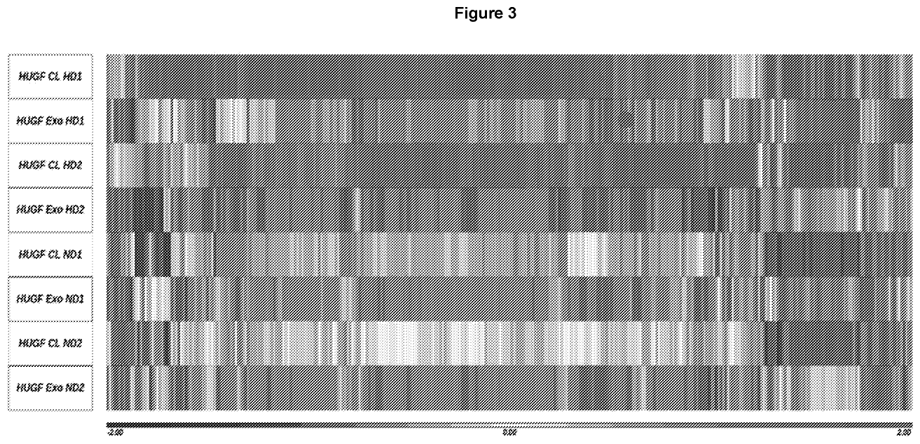

1155 miRNAs were identified in the entire dataset and reads normalised to Reads Per Million (RPM) with 2800 as maximal number of reads. FIG. 3 shows a heatmap (red signifying high abundance) of miRNA expression in cell lysate (CL) and exosomes (Exo) isolated from GF lines cultured under hypoxic (H) and normoxic (N) conditions for the different human donors (D1, D2). The pattern of miRNA abundance within GF cells and their exosomes is consistent between the samples. Notably, the GF cells contain a separate and unique set of MiRNAs to that of the exosomes produced by these cells. This differential miRNA expression is a consistent feature between the GF cells (D1 and D2) and their exosomes irrespective of the cells being cultured in normoxic or hypoxic conditions.

A total of 28 miRNAs were found to be more abundant in exosomes as compared to cell lysates (Table 1). Another 6 miRs were present within both cell lysates and exosomes. Of these 34 miRs, 14 are known to be proangiogenic and 8 have been described to be anti-angiogenic (4). Surprisingly, exosomes generated by gingival fibroblasts comprise a mix of pro-angiogenic and anti-angiogenic miRNAs species but with a dominance of pro-angiogenic miRNAs.

TABLE 1
mRNA levels in exosomes and cell lysates

| | | Exosomes | | Cell Lysate | |
|---|---|---|---|---|---|
| # | | Hypoxic | Normoxic | Hypoxic | Normoxic |
| 1 | 30c-5p | 2298 | 1806 | 111 | 31 |
| 2 | 181a-5p | 889 | 710 | 159 | 26 |
| 3 | 16-5p | 506 | 380 | 78 | 24 |
| 4 | 380-5p | 226 | 197 | 12 | 5.6 |
| 5 | 451a | 305 | 331 | 0 | 0 |
| 6 | 122-5p | 120 | 83 | 2 | 0.1 |
| 7 | 155-5p | 165 | 167 | 6 | 1.5 |
| 8 | 223-3p | 209 | 180 | 3.5 | 1 |
| 9 | 3160-3p | 24 | 18.5 | 1 | 0.2 |
| 10 | 139-5p | 10 | 13.6 | 0.5 | 0.5 |
| 11 | 144-3p | 54 | 70 | 0 | 0 |
| 12 | 142-3p | 22.7 | 25 | 0 | 0 |
| 13 | 150-5p | 18 | 56 | 0.2 | 0 |
| 14 | 150-3p | 44 | 25.7 | 2.8 | 0.8 |
| 15 | 10b-5p | 9.9 | 13.6 | 0 | 0 |
| 16 | 340-3p | 32.7 | 24 | 0.4 | 0 |
| 17 | 96-5p | 19.9 | 22.5 | 1.3 | 0.2 |
| 18 | 183-5p | 74 | 46.6 | 1.7 | 0.6 |
| 19 | 126-5p | 32 | 65 | 1.3 | 0 |
| 20 | 126-3p | 77 | 79 | 3.5 | 0.2 |
| 21 | 192-5p | 29.8 | 24 | 5.8 | 0.6 |
| 22 | 15a-5p | 38 | 40 | 58 | 38 |
| 23 | 18a-5p | 39.8 | 46 | 36 | 13 |
| 24 | 3529-3p | 45 | 8 | 15 | 6 |
| 25 | 195-5p | 31 | 38 | 13.6 | 4.4 |
| 26 | 19b-3p | 81 | 83 | 311 | 57 |
| 27 | 19a-3p | 39 | 70 | 224 | 41 |
| 28 | 218-5p | 59 | 51 | 11 | 3.6 |
| 29 | 25-3p | 18.5 | 19 | 31.6 | 3.6 |
| 30 | 93-5p | 109 | 88 | 177 | 47 |
| 31 | 106b-5p | 55.5 | 68 | 86 | 20 |
| 32 | 486-5p | 52.6 | 104 | 11 | 2 |
| 33 | 7-5p | 92.5 | 80 | 9.5 | 3 |
| 34 | 17-5p | 38 | 39 | 89 | 28 |
| 35 | 20a | 74 | 72 | 230 | 60 |

Amongst the pro-angiogenic miRNAs, miR-126 is 10-fold more abundant in exosomes as compared to cell lysate. miR-126 is well described to be one of the most potent angiomiRs that regulates the response of endothelial cells to VEGF (5-8).

The data show abundance of miR-125a-5p, miR-214-3p, miR-130a-3p, miR-204-5p, miR-126-3p, miR-30c-5p, miR-150-5p, miR-150-3p, miR-10b, miR-155, miR-25, miR-93, miR-19, miR-20, and miR-17.

Although the exact relative contribution of miRNAs to fibrosis and their targets are not fully known (9), as in other biological pathways, miRNAs can either enhance (pro) or inhibit (anti) the expression of proteins within the pathways and thus be pro-fibrotic or anti-fibrotic. Notably, one miRNA, miR-29 has emerged as the master regulator of fibrosis (10). This is a family of miRs and includes miR-29 a, b and c, which differ in one, two or three bases. Gingival fibroblast exosomes express miR-29a and miR-29b and notably, higher levels of miR-122 and miR-214, both described as anti-fibrotic miRNA species. These data suggest that GF-derived exosomes may also exhibit strong anti-fibrotic activities which could be exploited therapeutically.

### Endothelial Cell Proliferation.

The small RNA sequencing studies described above demonstrated GF exosomes contain a mix of micro-RNAs that promote angiogenesis. In vivo, angiogenesis is a well-regulated process that involves endothelial cell proliferation, migration and creation of new microvasculature. Endothelial cells regulate the passage of substances between the bloodstream and surrounding tissue, and secrete mediators that influence vascular hemodynamics, cell trafficking, and remodelling and formation of new blood vessels. Therefore, the present inventors assessed the ability of GF exosomes to directly promote endothelial cell proliferation in a cell-based assay.

The assay is based on a commercial product developed by Cellular Dynamics International (CDI), USA, to measure of proliferation of induced pluripotent stem cell (iPSC)-derived endothelial cells (iCells). iCells provide a reproducible and physiologically relevant source of cells for vascular disease modelling. The assay method used was adapted from the CDI application note and the iCell Endothelial Cell users guide. Cell proliferation was quantitated by measuring ATP with the CellTitre-Glo Cell Viability luminescent assay and an ATP standard curve.

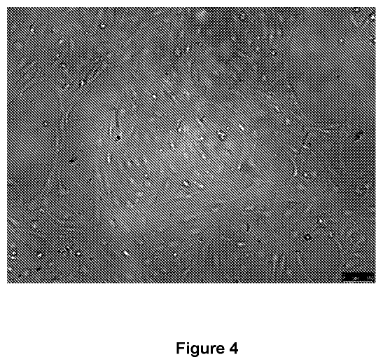

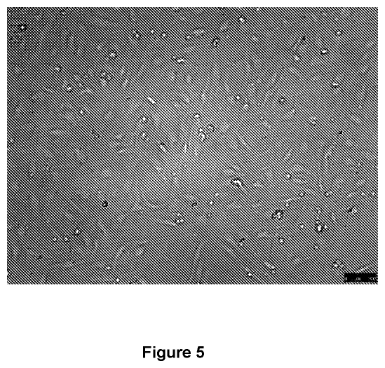

In brief, the iCells were thawed, cultured in humidified incubator with 5% CO₂ @ 37° C. and dissociated with Trypsin/EDTA. Cells were then plated out in a 96-well plate in 100 μL with between 1,600 and 2,000 cell per well. Cells were grown for a few days then placed under “controlled” starvation conditions where endothelial cell growth is slowed (or ceases completely) and the cells are primed to respond to a mitogen, like VEGF, provided in the full maintenance media. Agents for testing were added into cells, 24 hours after starvation and left for a further 3-5 days before proliferation was assessed by the ATP assay. Under these conditions the proliferative activity of the test agent was compared to that achieved by cells cultured under starvation conditions and to that achieved by addition of a known angiogenic stimulus such as VEGF. In all experiments, dilutions of the exosome samples were tested. An example of endothelial cells growing under these conditions when stimulated by GF exosomes (HuGF 1:10) and VEGF is shown in FIGS. 4 and 5.

The growth of the iCell endothelial cells was compared to that stimulated by VEGF and expressed as fold induction of cell proliferation as compared to VEGF. As is evident from FIGS. 4 and 5, the morphology, appearance and density of iCell endothelial cells was similar when cultured with VEGF or the test GF sample.

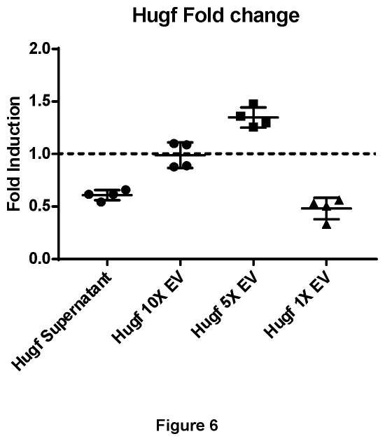

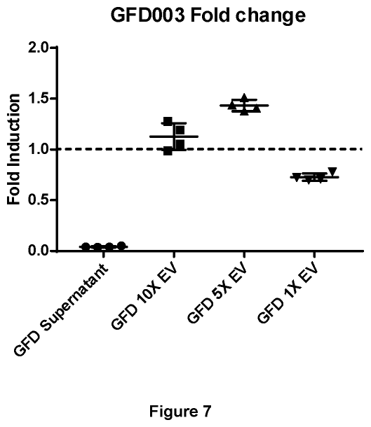

As shown in FIGS. 6 and 7, exosomes isolated from two GF cell lines (HuGF and GFD003) supported growth of iCell endothelial cells to a similar extent to VEGF (fold induction equivalent to 1.0). Notably, when exosome depleted supernatant was included, endothelial cells did not proliferate well. However, with both exosome samples from the two separate GF lines, the neat GF exosome preparation (1×) slightly inhibited iCell endothelial cell proliferation, as compared to the 1:5 (5× dilution) and 1:10 (10× dilution) diluted exosome sample, which may be attributed to co-purification of inhibitory contaminants by ultracentrifugation.

### Immortalisation of Gingival Fibroblasts

Gingival fibroblast (GF) cell lines that produce abundant, suitable exosomes for clinical use and that have been passaged at least 15 times (preferably 20 times) without loss of proliferative capacity can be selected for immortalization. The ability of cells to be passaged multiple times without any evidence of proliferative senescence is a way to select cells that can be immortalized by ectopic expression of the catalytic domain of human telomerase reverse transcriptase (hTERT) alone or by the overexpression of genes that regulate the cell cycle. Overexpression of the transcription factor c-Myc may also be used to immortalize GF cells.

Immortalization of selected GF cell lines may be achieved using methods for immortalization of human mesenchymal stromal/stem cells or bronchial epithelial cells that are well known in the art (11). One method involves ectopic expression of hTERT alone or in combination with overexpression of cyclin-dependent kinase-4 or 6 (Cdk4/6). In brief, this is done by viral vector-based delivery of hTERT (or Cdk4/6) with selection of transduced cells mediated by G418 or puromycin. Detailed methods are described in Ruben et al 2004 (11), Simonsen et al 2002 (12), and Liu et al 2013 (13). An alternative method is outlined in Benanti et al 2007 (14), which involves overexpression of c-Myc by transduction with a viral vector.

The retroviral or lentiviral plasmids for hTERT, Cdk4, and c-Myc can be obtained from commercial suppliers such as Addgene, Inc. (Watertown, MA). The plasmids are transfected into packaging cell lines (e.g., 293T cells or equivalent) by, for example, calcium phosphate precipitation and optionally selected with, for example, G418 at 400 μg/mL to 1000 μg/mL. Supernatant containing released viruses are used to infect GF cells in the presence of Polybrene (Sigma) or DEAE-dextran.

Polybrene stock is prepared to a concentration of 0.8 mg/ml or DEAE-dextran stock is prepared to a concentration of 0.1 or 0.2 mg/ml. Target GF cells are infected in a 6-well plate with 2 mL/well supernatant in the presence of 2-10 μg/ml Polybrene or 10 μg/ml DEAE-dextran. The target GF cells should preferably be 70-80% confluent at the time of infection and preferably in log-phase of growth.

Polybrene and DEAE-dextran are polycations that neutralize charge interactions to increase binding between the pseudoviral capsid and the cellular membrane. The skilled person will appreciate that the optimal concentration of Polybrene or DEAE-dextran depends on cell type and may need to be empirically determined (usually in the range of 2-10 μg/ml). Excessive exposure to Polybrene or DEAE-dextran (>12 hr) can be toxic to some cells.

The supernatant is removed from the wells 6-8 hours after infection (for Polybrene) or 2 hours after infection (for DEAE-dextran), after which the cells can be re-infected with a further 2 mL of supernatant containing released viruses in the presence of Polybrene (Sigma) or DEAE-dextran, or allowed to recover in fresh media for 72 hours, optionally followed by drug selection.

For lentiviral vector, one infection typically works well for most target cells. The skilled person will appreciate that the lentiviral vector can be diluted with fresh complete growth medium (at, for example, 1:1) if cytotoxicity is a problem.

After recovery for 72 hours, the cells are sub-cultured into 2×100 mm dishes and cultured under drug selection for 10-15 days for stable cell-line generation.

For the EGFP control retrovirus in the method of (11), the selection marker is Puromycin; the selection concentration is typically between 1-10 μg/ml. To confirm immortalization, cells transduced with hTERT or c-Myc viral vectors may be passaged beyond the number of population doublings required for control cells (transduced with empty or EGFP retrovirus or lentivirus) to reach senescence.

The cells are subsequently screened for hTERT or c-Myc expression. GF cells expressing hTERT or c-Myc are then be expanded and the exosomes from these cells analysed to determine characteristics, molecular cargo and functional activity in relevant bioassays.

### REFERENCES

1. Ge S, Mrozik K M, Menicanin D, Gronthos S & PM Bartold. Isolation and characterisation of mesenchymal stem cell-like cells from healthy and inflamed gingival tissue: potential use for clinical therapy. Regen Med. (2012) 7 (6): 819-832.
2. Silvestro S. et al. Extracellular vesicles derived from human gingival mesenchymal stem cells: A transcriptional analysis. Genes (2020) 11 (2): 118.
3. Lesley Cheng and Andrew F. Hill. Small RNA library construction for exosomal RNA from biological samples for the Ion Torrent PGM and Ion S5 System. Exosomes and Microvesicles: Methods and Protocols, Methods in Molecular Biology, Vol. 1545, DOI 10.1007/978-1-4939-6728-5_6.
4. Landskroner-Eiger S, Moneke I, Sessa WC. miRNAs as modulators of angiogenesis. Cold Spring Harbor Perspectives in Medicine 2013:3:a006643.
5. Shusheng Wang and Eric Olson. AngiomiRs-Key regulators of angiogenesis. Current Opinion in Genetics & Development (2009) 19:205-211.
6. Fish J E et al. miR-126 regulates angiogenic signaling and vascular integrity. Dev. Cell (2008) 15:272-284.
7. Harris T A et al. MicroRNA-126 regulates endothelial expression of vascular cell adhesion molecule-1. Proc. Natl. Acad. Sci. (208) 105:1516-121.
8. Wang S et al. The endothelial-specific microRNA miR-126 governs vascular I ntegrity and angiogenesis. Dev. Cell (2008) 15:261-271.
9. Steven O'Reilly. MicroRNAs in fibrosis: opportunities and challenges. Arthritis Research & Therapy (2016) 18:11.
10. Van Rooij E et al. Dysregulation of microRNAs after myocardial infarction reveals a role of miR-29 in cardiac fibrosis. Proc. Natl. Acad. Sci. USA. (2008): 105:13027-32.
11. Ruben D. Ramirez, Shelley Sheridan, Luc Girard, Mitsuo Sato, et al. Immortalization of Human Bronchial Epithelial Cells in the Absence of Viral Oncoproteins. Cancer Research (2004) 64:9027-9034.
12. Simonsen J L, Rosada C, Serakinca N et al. Telomerase expression extends the proliferative life-span and maintains the osteogenic potential of human bone marrow stromal cells. Nature Biotechnology (2002) 20:592-596.
13. Liu T M, Ng W M, Tan W S, et al. Molecular basis of immortalisation of human mesenchymal stem cells by combination of p53 knockdown and human telomeres reverse transcriptase overexpression. Stem Cells and Development (2013) 22:268-277.
14. Benanti J A et al., Epigenetic down-regulation of ARF expression is a selection step in immortalization of human fibroblasts by c-Myc. Mol. Cancer Res. (2007) 5 (11): 1181-1189.

1. A population of gingival fibroblast derived extracellular vesicles.

2. The population according to claim 1, wherein the extracellular vehicles are exosomes.

3. The population according to claim 1 or claim 2, wherein the extracellular vesicles are allogeneic.

4. The population according to any one of claims 1 to 3, wherein the extracellular vesicles are derived from primary gingival fibroblasts, or from gingival fibroblasts expanded in culture, or from an immortalized gingival fibroblast cell line.

5. The population according to any one of claims 1 to 4, wherein the extracellular vesicles comprise one or more microRNAs selected from miR-125a-5p, miR-214-3p, miR-130a-3p, miR-204-5p, miR-126-3p and miR-30c-5p.

6. The population according to any one of claims 1 to 4, wherein the extracellular vesicles comprise one or more microRNAs selected from miR-125a-5p, miR-214-3p, miR-130a-3p, miR-204-5p, miR-126-3p, miR-30c-5p, miR-155-5p, miR-150-5p, miR-150-3p, miR-10b-5p, miR-126-5p, miR-25-3p, and miR-93-5p.

7. The population according to any one of claims 1 to 4, wherein the extracellular vesicles comprise one or more microRNAs selected from miR-125a-5p, miR-214-3p, miR-130a-3p, miR-204-5p, miR-126-3p, miR-30c-5p, miR-155-5p, miR-150-5p, miR-150-3p, miR-10b-5p, miR-126-5p, miR-25-3p, miR-93-5p, miR-19b-3p, miR-20a-5p, miR-19a-3p, and miR-17-5p.

8. The population according to any one of claims 1 to 4, wherein the extracellular vesicles comprise one or more microRNAs selected from miR-30c-5p, miR-155-5p and miR150-5p.

9. The population according to any one of claims 1 to 8, wherein the extracellular vesicles comprise one or more microRNAs selected from miR181a-5p, miR16-5p, miR-451a, miR-144-3p, miR-96-5p, miR-183-5p, and miR-7-5p.

10. The population according to any one of claims 1 to 9, wherein the extracellular vesicles comprise one or more microRNAs selected from miR-380-5p, miR-122-5p, miR-223-3p, miR-3160-3p, miR-139-5p, miR-142-3p, miR-340-3p, miR-192-5p, miR-18a-5p, miR-3529-3p, miR-195-5p, miR-218-5p, miR-106b-5p, and miR-486-5p.

11. The population according to any one of claims 5 to 10, wherein the ratio of extracellular vesicle microRNAs to cell lysate microRNAs is at least 2:1.

12. The population according to claim 11, wherein the ratio of extracellular vesicle microRNAs to cell lysate microRNAs is at least 5:1 for one or more microRNAs selected from miR-130a, miR-17-5p, miR-19a-3p, miR-20a-5p, miR-19b-3p, miR-7-5p, miR-486-5p, miR-106b-5p, miR-93-5p, miR-25-3p, miR-218-5p, miR-195-5p, miR-3529-3p, miR-18a-5p, miR-192-5p, miR-126-3p, miR-126-5p, miR-183-5p, miR-96-5p, miR-340-3p, miR-10b-5p, miR-150-3p, miR-150-5p, miR-142-3p, miR-144-3p, miR-139-5p, and miR-3160-3p.

13. The population according to claim 11, wherein the ratio of extracellular vesicle microRNAs to cell lysate microRNAs is at least 10:1 for one or more microRNAs selected from miR-223-3p, miR-155-5p, miR-122-5p, miR-451a, and miR-380-5p.

14. The population according to claim 11, wherein the ratio of extracellular vesicle microRNAs to cell lysate microRNAs is at least 100:1 for one or more microRNAs selected from miR-16-5p, miR-181a-5p, and miR-30c-5p.

15. The population according to any one of claims 1 to 14, wherein the extracellular vesicles are derived from gingival fibroblasts cultured under hypoxic or normoxic conditions.

16. The population according to any of one of claims 1 to 15, wherein at least 50% of the extracellular vesicles have a particle size of about 30 nm to about 150 nm.

17. The population according to any one of claims 1 to 16, wherein the population promotes angiogenesis.

18. The population according to any one of claims 1 to 16, wherein the population reduces fibrosis.

19. The population according to any one of claims 1 to 16, wherein the population slows the rate of progression of fibrosis.

20. A composition comprising the population of extracellular vesicles according to any one of claims 1 to 19, and one or more pharmaceutically acceptable carriers or excipients.

21. The population according to any one of claims 1 to 19, or the composition of claim 20 for use in therapy.

22. The population or composition according to claim 21, wherein the composition is for use in the promotion of wound healing.

23. The population or composition according to claim 21, wherein the composition is for use in the treatment or prevention of ischemic diseases.

24. The population or composition according to claim 21, wherein the composition is for use in the treatment or prevention of fibrotic diseases.

25. A method for promoting wound healing in a subject, the method comprising administering an effective amount of gingival fibroblast derived extracellular vesicles to the wound.

26. A method for promoting tissue repair and/or regeneration in a subject comprising administering an effective amount of gingival fibroblast derived extracellular vesicles to the subject.

27. A method of treating or preventing an ischemic disease in a subject comprising administering an effective amount of gingival fibroblast derived extracellular vesicles to the subject.

28. A method of treating or preventing fibrotic disease in a subject comprising administering an effective amount of gingival fibroblast derived extracellular vesicles to the subject.

29. A population of extracellular vesicles, wherein the extracellular vesicles comprise one or more microRNAs selected from miR-30c-5p, miR-10b-5p, miR-126-5p, miR-25-3p, miR-93-5p, miR-19b-3p, miR-20a, miR-19a-3p, and miR-17-5p.

30. The population according to claim 29, wherein the extracellular vesicles comprise one or more microRNAs selected from miR16-5p, miR-451a, and miR-7-5p.

31. The population according to claim 29 or claim 30, wherein the extracellular vesicles comprise one or more microRNAs selected from miR-139-5p, miR-142-3p, and miR-18a-5p.

32. A population of extracellular vesicles, wherein the extracellular vesicles comprise one or more microRNAs selected from miR-130a-3p, miR-126-3p, miR-150-5p, miR-10b-5p, and miR-126-5p.

33. The population according to claim 32, wherein the extracellular vesicles comprise one or more microRNAs selected from miR-25-3p, miR-93-5p, miR-19b-3p, miR-20a-5p, miR-19a-3p, and miR-17-5p.

34. The population according to claim 32 or claim 33, wherein the extracellular vesicles comprise one or more microRNAs selected from miR-451a and miR-144-3p.

35. The population according to any one of claims 32 to 34, wherein the extracellular vesicles comprise one or more microRNAs selected from miR-139-5p, miR-142-3p, miR-18a-5p, miR-3529-3p, miR-195-5p, and miR-486-5p.

36. The population according to any one of claims 33 to 35, wherein the extracellular vesicles comprise miR-106b-5p.

37. The population according to any of one of claims 29 to 36, wherein at least 50% of the extracellular vesicles have a particle size of about 30 nm to about 150 nm.

38. The population according to any one of claims 29 to 37, wherein the population promotes angiogenesis.

39. The population according to any one of claims 29 to 37, wherein the population reduces fibrosis.

40. The population according to any one of claims 29 to 37, wherein the population slows the rate of progression of fibrosis.

41. A composition comprising the population of extracellular vesicles according to any one of claims 29 to 40, and one or more pharmaceutically acceptable carriers or excipients.

42. The population according to any one of claims 29 to 40, or the composition of claim 41 for use in therapy.

43. The population or composition according to claim 42, wherein the composition is for use in the promotion of wound healing.

44. The population or composition according to claim 42, wherein the composition is for use in the treatment or prevention of ischemic diseases.

45. The population or composition according to claim 42, wherein the composition is for use in the treatment or prevention of fibrotic diseases.

46. A method for promoting wound healing in a subject, the method comprising administering an effective amount of a population according to any one of claims 29 to 40 to the subject.

47. A method for promoting tissue repair and/or regeneration in a subject comprising administering an effective amount of a population according to any one of claims 29 to 40 to the subject.

48. A method of treating or preventing an ischemic disease in a subject comprising administering an effective amount of a population according to any one of claims 29 to 40 to the subject.

49. A method of treating or preventing fibrotic disease in a subject comprising administering an effective amount of a population according to any one of claims 29 to 40 to the subject.
